(19) United States
(12) Patent Application Publication
(10) Pub. No.: US 2026/0210893 A1
(43) Pub. Date: Jul. 23, 2026

(54) ORGANIC HYDROGEN SENSOR

(71) Applicant: King Abdullah University of Science and Technology, Thuwal (SA)

(72) Inventors: Thomas ANTHOPOULOS, Thuwal (SA); Hendrik Andreas FABER, Thuwal (SA); Suman MANDAL, Thuwal (SA)

(21) Appl. No.: 19/142,714

(22) PCT Filed: Mar. 14, 2024
(86) PCT No.: PCT/IB2024/052502

§ 371 Date: Jun. 24, 2025

Related U.S. Application Data
(60) Provisional application No. 63/451,977, filed on Mar. 14, 2023.

(51) Int. Cl. G01N 27/12; G01N 33/00

(52) U.S. Cl. CPC G01N 27/126; G01N 33/005

(57) ABSTRACT

A hydrogen sensor includes a substrate, a first terminal, and a second terminal. The first and second terminal are disposed on the substrate and separated by a gap. An organic semiconductor is electrically coupled to the first terminal and the second terminal. Exposure of the organic semiconductor to hydrogen de-dopes the organic semiconductor of oxygen. Conductivity of the organic semiconductor thereby decreases upon exposure to hydrogen.

### BACKGROUND

Hydrogen (H₂) is an efficient and abundant clean source of energy exhibiting excellent properties such as high energy density and light weight. These attributes make it suitable for various applications, including energy, transportation, petroleum refining, defense, space, agriculture, medicine, etc. However, hydrogen is highly combustible, having a flammability point of about 4 vol % of H₂ in the air. Thus, hydrogen poses serious safety concerns during H₂ production, storage, and usage. There exists an increasing demand for hydrogen sensors that are highly sensitive, have ultrafast response time, are low cost, energy efficient, and/or operable under ambient (room temperature) conditions.

Resistive-type hydrogen sensors and/or transistor-type hydrogen sensors include an inorganic semiconductor which is responsive to hydrogen exposure. The resistance of the inorganic semiconductor decreases (i.e., conductivity increases) when hydrogen interacts with the inorganic semiconductor. The resistive-type/transistor type hydrogen sensor monitors the change in resistance, i.e., one or more characteristics of an electrical signal between two electrodes is modified due to the decreasing resistance of the inorganic semiconductor, and thus, detects the presence of hydrogen based on one or more characteristics of the electrical signal. However, inorganic semiconductors generally have poor response time, low sensitivity, poor recovery time, and require working at high temperatures. Therefore, there exists a need for a high sensitivity, ultrafast response time, rapid recovery time, and/or room-temperature hydrogen sensor.

### OVERVIEW

According to some embodiments, a hydrogen sensor includes a substrate, a first terminal, and a second terminal. The first and second terminal are disposed on the substrate and separated by a gap. An organic semiconductor is electrically coupled to the first terminal and the second terminal. Exposure of the organic semiconductor to hydrogen de-dopes the organic semiconductor of oxygen. Conductivity of the organic semiconductor thereby decreases upon exposure to hydrogen.

According to some embodiments, a system for detecting hydrogen includes an organic hydrogen sensor. The organic hydrogen sensor includes a first terminal and a second terminal physically separated from the first terminal by a gap. An organic semiconductor is electrically coupled to the first terminal and the second terminal. A processing circuit is electrically coupled to the first terminal and the second terminal. The processing circuit transmits an input signal to one of the first terminal and the second terminal, and receives an output signal from one of the first terminal and the second terminal.

These and other examples and features of the present devices, systems, and methods will be set forth, at least in part, in the following Detailed Description. This Overview is intended to provide non-limiting examples of the present subject matter-it is not intended to provide an exclusive or exhaustive explanation. The Detailed Description below is included to provide further information about the present devices, systems, and methods.

### BRIEF DESCRIPTION OF DRAWINGS

This written disclosure describes illustrative embodiments that are non-limiting and non-exhaustive. Reference is made to illustrative embodiments that are depicted in the figures, in which:

FIG. 1A is a diagrammatic view of an organic hydrogen sensor, according to some embodiments.

FIG. 1B is a diagrammatic view of an organic hydrogen sensor, according to some embodiments.

FIG. 1C is a diagrammatic view of an organic hydrogen sensor, according to some embodiments.

FIG. 1D is a diagrammatic view of an organic hydrogen sensor, according to some embodiments.

FIG. 2A is a diagrammatic side view of an organic hydrogen sensor, according to some embodiments.

FIG. 2B is a diagrammatic side view of an organic hydrogen sensor, according to some embodiments.

FIG. 3A is a schematic diagram of an exemplary circuit to determine an unknown resistance of an organic hydrogen sensor, according to some embodiments.

FIG. 3B is a schematic diagram of an exemplary circuit to determine an unknown resistance of an organic hydrogen sensor, according to some embodiments.

FIG. 3C is a schematic diagram of an exemplary circuit to determine an unknown resistance of an organic hydrogen sensor, according to some embodiments.

FIG. 4A is a diagrammatic view of a bottom-gate top-contact (BGTC) transistor-type organic hydrogen sensor, according to some embodiments.

FIG. 4B is a diagrammatic view of a bottom-gate bottom-contact (BGBC) transistor-type organic hydrogen sensor, according to some embodiments.

FIG. 4C is a diagrammatic view of a top-gate bottom-contact (TGBC) transistor-type organic hydrogen sensor, according to some embodiments.

FIG. 4D is a diagrammatic view of a top-gate top-contact (TGTC) transistor-type organic hydrogen sensor, according to some embodiments.

FIG. 5A is a plot of voltage (V) versus current (μA) as hydrogen concentration (ppm) changes from an exemplary organic hydrogen sensor having electrodes coated with an organic semiconductor, according to some embodiments.

FIG. 5B is a plot of current variation (A) with time(s) measured at a constant applied voltage of 0.5V from an exemplary organic hydrogen sensor having electrodes coated with an organic semiconductor, according to some embodiments.

FIG. 5C is a plot of current (A) versus hydrogen concentration (H₂ ppm) with a constant applied voltage of 0.5V from an exemplary organic hydrogen sensor having electrodes coated with an organic semiconductor, according to some embodiments.

FIG. 5D is a plot of real-time(s) current changes (A) at different hydrogen concentrations (ppm) from an exemplary organic hydrogen sensor having electrodes coated with an organic semiconductor, according to some embodiments.

FIG. 5E is a plot of the change in resistance (ΔR/R₀) of the organic semiconductor versus hydrogen concentration (H₂ ppm) from an exemplary organic hydrogen sensor having electrodes coated with an organic semiconductor, according to some embodiments.

FIG. 5F is a plot of the change in resistance (ΔR/R₀) of the organic semiconductor versus hydrogen concentration (H₂ ppm) at different applied voltages (0.1V to 1.0V) from an exemplary organic hydrogen sensor having electrodes coated with an organic semiconductor, according to some embodiments.

FIG. 6A is a plot of normalized resistance versus time(s) upon exposure of the organic hydrogen sensor to 500 ppm of H₂ from an exemplary organic hydrogen sensor having electrodes coated with an organic semiconductor, according to some embodiments.

FIG. 6B is a bell curve of response times of the hydrogen sensor in response to exposure of 500 ppm of H₂ from an exemplary organic hydrogen sensor having electrodes coated with an organic semiconductor, according to some embodiments.

FIG. 6C is a bell curve of recovery times of the hydrogen sensor in response to exposure of 500 ppm of H₂ from an exemplary organic hydrogen sensor having electrodes coated with an organic semiconductor, according to some embodiments.

FIG. 6D is a plot 630 of continuous H₂ measurements for 2000 cycles from an exemplary organic hydrogen sensor having electrodes coated with an organic semiconductor, according to some embodiments.

FIG. 7A is a magnified diagrammatic view of a humidity sensor, according to some embodiments.

FIG. 7B is a magnified diagrammatic view of a humidity sensor, according to some embodiments.

FIG. 8A is an exemplary process of assembling a nanogap between electrodes, according to some embodiments.

FIG. 8B is an exemplary process of assembling a nanogap between electrodes, according to some embodiments.

FIG. 8C is an exemplary process of assembling a nanogap between electrodes, according to some embodiments.

FIG. 8D is an exemplary process of assembling a nanogap between electrodes, according to some embodiments.

FIG. 8E is an exemplary process of assembling a nanogap between electrodes, according to some embodiments.

FIG. 8F is an exemplary process of assembling a nanogap between electrodes, according to some embodiments.

### DETAILED DESCRIPTION

According to some embodiments, this disclosure relates to devices, systems, and methods for sensing hydrogen (H₂) with an organic hydrogen sensor. The organic hydrogen sensor includes an organic semiconductor (OSC) and a catalytic material. Upon exposure to hydrogen (H₂) in the air, the catalytic material splits molecular hydrogen (H₂) into atomic hydrogen (H). The organic semiconductor is de-doped (deoxidized) by the atomic hydrogen (H) which thereby decreases the conductivity of the organic semiconductor. Thus, the conductivity of the organic semiconductor is indicative of the hydrogen (H₂) concentration in the air—the resistance of the organic semiconductor increases with increasing hydrogen (H₂) concentration. The organic hydrogen sensor includes one or more terminals for sensing the conductivity of the organic semiconductor (e.g., electrodes or gate/source/drain) to thereby determine the concentration of hydrogen (H₂) in the air.

The organic semiconductor is a p-type (positive charge transporting) semiconductor. Organic semiconductors are doped upon exposure to oxygen, i.e., the holes of the organic semiconductor receive electrons from oxygen which dopes the organic semiconductor. Upon exposure to air, the organic semiconductor of the organic hydrogen sensor becomes oxygen doped. Doping of the organic semiconductor increases conductivity (and decreases resistivity) of the organic semiconductor. Exposing the oxygen-doped organic semiconductor to hydrogen de-dopes (deoxidizes) the organic semiconductor. In other words, hydrogen interacts with oxygen to decouple the (oxygen) electron from the hole of the organic semiconductor. The de-doping (or deoxidizing) of the organic semiconductor is selective to hydrogen—no other element/gas will decouple the (oxygen) electron from the hole of the organic semiconductor.

The catalytic material is configured to split molecular hydrogen (H₂) into atomic hydrogen (H). Hydrogen gas present within the air is in the form H₂. However, the molecular hydrogen/hydrogen gas (H₂) will not decouple the oxygen electron from the hole of the organic semiconductor. Therefore, the catalytic material splits molecular hydrogen (H₂) into atomic hydrogen (H) by breaking the dipole-dipole bond between the hydrogen atoms. Atomic hydrogen (H) will decouple the oxygen electron from the hole of the organic semiconductor.

The catalytic material includes one or more of palladium (Pd), platinum (Pt), gold (Au), and/or other hydrogen reactive catalytic materials. In some embodiments, the catalytic material is in the form of nanostructures (nanoparticles, nanowires, nanorods, nano-pyramids, nano-cubes, etc.). The catalytic nanostructures can be dispersed within the organic semiconductor and/or can be deposited on a surface of the organic semiconductor. In some embodiments, the one or more elements for sensing the conductivity of the organic semiconductor (e.g., electrodes or gate/source/drain) are formed from the catalytic material.

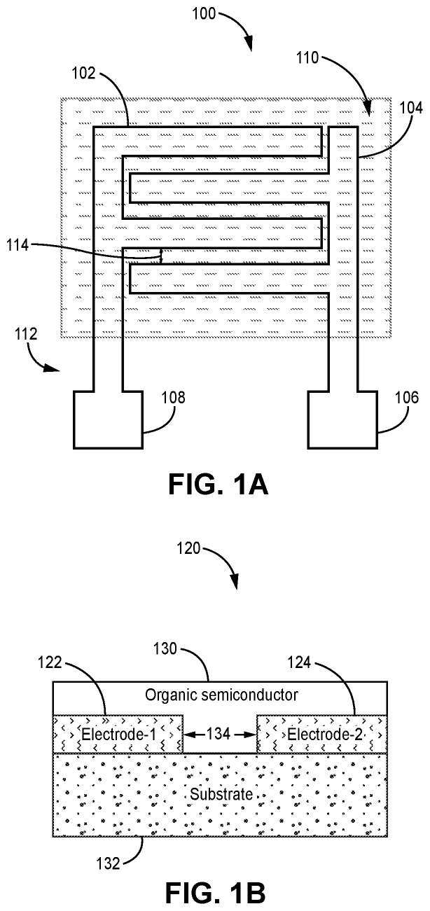

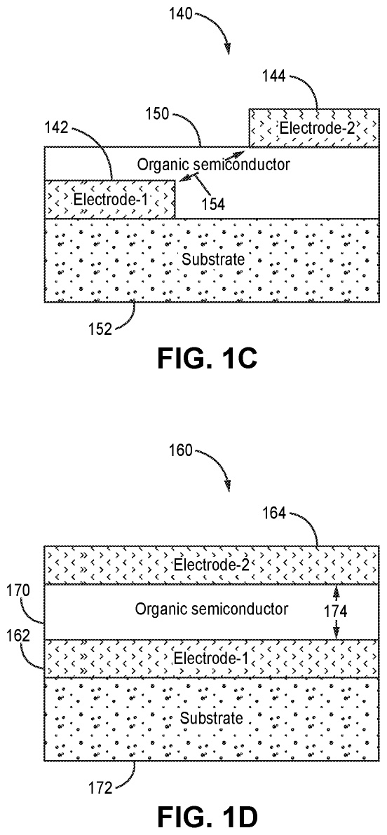

FIG. 1A is a diagrammatic view of an organic hydrogen sensor 100, according to some embodiments. The organic hydrogen sensor 100 includes a first electrode 102, a second electrode 104, a first terminal 106, a second terminal 108, an organic semiconductor 110, a substrate 112, and a gap 114 between the first electrode 102 and the second electrode 104. The first electrode 102 is directly coupled with the first terminal 106 and the second electrode 104 is directly coupled with the second terminal 108. Electrical signals may be sent to and/or received from the first electrode 102 and the second electrode 104 via the first terminal 106 and second terminal 108, respectively. The first terminal 106 and the second terminal 108 can be electrically coupled with a processor/electronic control unit (ECU) to send/receive electrical signals to the first electrode 102 and the second electrode 104. For instance, an electrical signal generated by the ECU is sent to the first electrode 102. The electrical signal travels from the first electrode 102, through the organic semiconductor 110, to the second electrode 104 across the gap 114. The received electrical signal is communicated from the second electrode 104 to the ECU.

In some embodiments, the first electrode 102 and the second electrode 104 are positioned on the organic semiconductor 110 such that air can directly interact with the first electrode 102 and the second electrode 104. In other embodiments (such as FIG. 1B), the first electrode 102 and/or the second electrode 104 is positioned under the organic semiconductor 110 such that air cannot directly interact with the first electrode 102 and the second electrode 104. In some embodiments (such as FIGS. 1C-D), the first electrode 102 is positioned above or below the second electrode 104.

In some embodiments, the organic semiconductor 110 includes a p-type (positive charge transporting) semiconductor dopeable upon exposure to oxygen, i.e., the holes of the organic semiconductor 110 receive electrons from oxygen which dopes the organic semiconductor. Upon exposure to air, the organic semiconductor 110 of the organic hydrogen sensor 100 becomes oxygen doped. Doping of the organic semiconductor 110 increases conductivity (and decreases resistivity) of the organic semiconductor 110. Exposing the oxygen-doped organic semiconductor 110 to hydrogen de-dopes (deoxidizes) the organic semiconductor 110. In other words, hydrogen interacts with oxygen to decouple the (oxygen) electron from the hole of the organic semiconductor 110. The de-doping (or deoxidizing) of the organic semiconductor 110 is selective to hydrogen—no other element/gas will decouple the (oxygen) electron from the hole of the organic semiconductor 110.

In some embodiments, the organic semiconductor 110 is formed of an organic material, including for example, pi-bonded molecules or polymers made up by carbon and hydrogen atoms and/or heteroatoms such as nitrogen, sulfur and oxygen. The particular material of the organic semiconductor 110 may be chosen, so long as the material includes hole-transporting p-type qualities with high ionization potential. The hole-transporting p-type qualities with high ionization potential is important, as it enables oxygen doping of the organic semiconductor 110 and enables de-doping upon exposure to hydrogen. In some embodiments, the organic semiconductor 110 is formed from conjugated polymers such as DPP-DTT, C₁₆IDT-BT, and/or poly(3-hexylthiophene-2,5-diyl). In some embodiments, the organic semiconductor 110 is formed of organic heterocyclic conjugated materials that contain conjugated segments, aromatic rings, and/or thiophene, such as alkylmonothiophenes, aryl/heteroarylmonothiophenes, benzothiophenes, condensed cyclic thiophenes, halomonothiophenes, monothiophenes, oligothiophenes, polythiophenes among many other known to those skilled in the art of organic semiconductors. Other types of conjugated polymers and/or heterocyclic conjugated materials are possible-so long as they are oxygen dopable and de-dope upon exposure to hydrogen. In some embodiments, the organic semiconductor 110 is formed as a thin-film/layer. In some embodiments, the organic semiconductor 110 includes nanostructures and/or blends of various organic or inorganic materials.

The organic hydrogen sensor 100 includes a catalytic material. In some embodiments, the catalytic material is in the form of nanostructures (nanoparticles, nanowires, nanorods, nano-pyramids, nano-cubes, etc.) dispersed within the organic semiconductor 110 and/or deposited on a surface of the organic semiconductor 110. In some embodiments, the first electrode 102 and the second electrode 104 are formed of the catalytic material. The catalytic material includes one or more of palladium (Pd), platinum (Pt), gold (Au), and/or other hydrogen reactive catalytic materials, including for example Zinc Oxide (ZnO), Molybdenum trioxide (MoO₃), Titanium dioxide (TiO₂), Niobium pentoxide (Nb₂O₅), Tin(IV) oxide (SnO₂), Tungsten Oxide (W₂O₃) and Indium(III) oxide (In₂O₃).

The catalytic material is configured to split molecular hydrogen (H₂) into atomic hydrogen (H). Hydrogen gas present within the air is in the form H₂. However, the molecular hydrogen/hydrogen gas (H₂) will not decouple the oxygen electron from the hole of the organic semiconductor 110. Therefore, the catalytic material splits molecular hydrogen (H₂) into atomic hydrogen (H) by breaking the dipole-dipole bond between the hydrogen atoms. Atomic hydrogen (H) will decouple the oxygen electron from the hole of the organic semiconductor.

The organic hydrogen sensor 100 shown in FIG. 1A is a resistive-type hydrogen sensor, i.e., an electrical signal is applied to a first electrode 102 and measured by a second electrode 104 (or vice-versa). The measured electrical signal is compared to the applied electrical signal, and one or more conductive properties (e.g., resistance) of the organic semiconductor 110 is determined. For example, one or more characteristics of the measured electrical signal (i.e., output signal) include voltage, current, resistance, capacitance and/or impedance. In some embodiments, the first electrode 102 and the second electrode 104 are interdigitated metal electrodes (IDEs) where a resistance and/or capacitance of a material (e.g., the organic semiconductor 110) can be calculated between electrode features.

For example, if the organic hydrogen sensor 100 is exposed to hydrogen gas (H₂), the catalytic material splits the molecular hydrogen (H₂) into atomic hydrogen (H). The atomic hydrogen (H) interacts with the oxygen-doped organic semiconductor 110, and de-dopes the organic semiconductor 110. The organic semiconductor 110 exhibits decreased conductivity due to the de-doping—the degree/magnitude of conductivity is dependent on the hydrogen gas (H₂) concentration in the air. An electrical signal such as a voltage or current is applied to the first electrode 102 (in some case, via the first terminal 106). The electrical signal travels across the organic semiconductor and is received by the second electrode 104. The voltage and/or current of the electrical signal is measured, and thereby, the resistance (V=IR) of the organic semiconductor 110 is determined.

In some embodiments, the substrate 112 includes a glass material, a dielectric material, a printed circuit board (PCB) substrate, a flexible substrate such as PET, PEN, etc., or other materials such as paper or nano-cellulose.

FIG. 1B is a diagrammatic view of an organic hydrogen sensor 120, according to some embodiments. The organic hydrogen sensor 120 includes a first electrode 122, a second electrode 124, an organic semiconductor 130, a substrate 132, and a gap 134 between the first electrode 122 and the second electrode 124. The organic semiconductor 130 includes any and/or all features of the organic semiconductor 110 described above in reference to FIG. 1A. The organic hydrogen sensor 120 shown in FIG. 1B is a resistive-type hydrogen sensor, i.e., an electrical signal is applied to a first electrode 122 and measured by a second electrode 124 (or vice-versa). The measured electrical signal is compared to the applied electrical signal, and one or more conductive properties (e.g., resistance) of the organic semiconductor 130 is determined.

In some embodiments, the organic semiconductor 130 is positioned to encapsulate the first electrode 122 and the second electrode 124. The air/atmosphere being measured by the organic hydrogen sensor 120 interacts with the organic semiconductor 130, and in some embodiments, the air/atmosphere cannot directly interact with the first electrode 122 and the second electrode 124.

FIG. 1C is a diagrammatic view of an organic hydrogen sensor 140, according to some embodiments. The organic hydrogen sensor 140 includes a first electrode 142, a second electrode 144, an organic semiconductor 150, a substrate 152, and a gap 154 between the first electrode 142 and the second electrode 144. The organic semiconductor 150 includes any and/or all features of the organic semiconductor 110 described above in reference to FIG. 1A. The organic hydrogen sensor 140 shown in FIG. 1C is a resistive-type hydrogen sensor, i.e., an electrical signal is applied to a first electrode 142 and measured by a second electrode 144 (or vice-versa). The measured electrical signal is compared to the applied electrical signal, and one or more conductive properties (e.g., resistance) of the organic semiconductor 140 is determined.

In some embodiments, the organic semiconductor 150 is positioned to encapsulate the first electrode 142, i.e., air/atmosphere being measured by the organic hydrogen sensor 140 interacts with the organic semiconductor 150 but cannot directly interact with the first electrode 142. The organic semiconductor 150 is positioned below the second electrode 144 to allow the air/atmosphere being measured by the organic hydrogen sensor 140 to directly interact with the second electrode 144.

FIG. 1D is a diagrammatic view of an organic hydrogen sensor 160, according to some embodiments. The organic hydrogen sensor 160 includes a first electrode 162, a second electrode 164, an organic semiconductor 170, a substrate 172, and a gap 174 between the first electrode 162 and the second electrode 164. The organic semiconductor 170 includes any and/or all features of the organic semiconductor 110 described above in reference to FIG. 1A. The organic hydrogen sensor 160 shown in FIG. 1C is a resistive-type hydrogen sensor, i.e., an electrical signal is applied to a first electrode 162 and measured by a second electrode 164 (or vice-versa). The measured electrical signal is compared to the applied electrical signal, and one or more conductive properties (e.g., resistance) of the organic semiconductor 160 is determined.

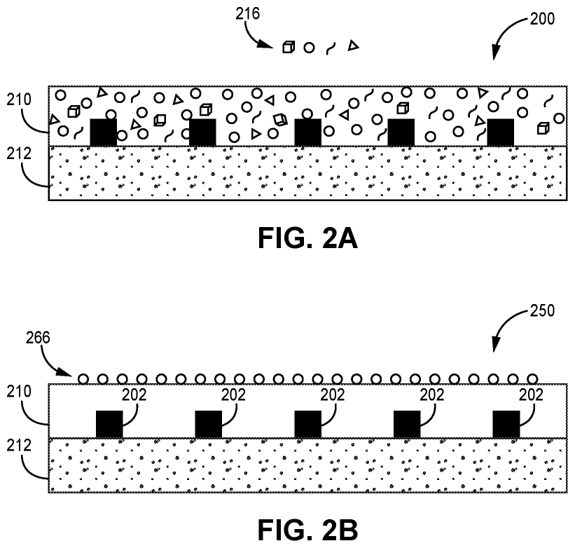

FIG. 2A is a diagrammatic side view of an organic hydrogen sensor 200, according to some embodiments. The organic hydrogen sensor 200 includes a plurality of electrodes 202, an organic semiconductor 210, a substrate 212, and catalytic metal nanostructures 216. The organic semiconductor 210 includes any and/or all features of the organic semiconductor 110 described above in reference to FIG. 1A. The catalytic metal nanostructures 216 are dispersed within the organic semiconductor 210 in a random, substantially uniform distribution. In some embodiments, the catalytic metal nanostructures 216 includes one or more catalytic materials, including for example, palladium (Pd), platinum (Pt), gold (Au), other hydrogen reactive catalytic materials, including for example Zinc Oxide (ZnO), Molybdenum trioxide (MoO₃), Titanium dioxide (TiO₂), Niobium pentoxide (Nb₂O₅), Tin(IV) oxide (SnO₂), Tungsten Oxide (W₂O₃) and Indium(III) oxide (In₂O₃), and/or combinations thereof.

FIG. 2B is a diagrammatic side view of an organic hydrogen sensor 250, according to some embodiments. The organic hydrogen sensor 250 includes the plurality of electrodes 202, the organic semiconductor 210, a substrate 212, and catalytic nanostructure layer 266. In some embodiments, the catalytic nanostructure layer 266 is pre-synthesized or formed spontaneously on a surface of the organic semiconductor 210. For example, deposition of an ultra-thin layer of catalytic metal, such as Pt or Pd, atop the organic semiconductor 210 forms the catalytic nanostructure layer 266 that are catalytic for hydrogen. Such configuration would eliminate the requirement for a catalytic metal as the electrode material.

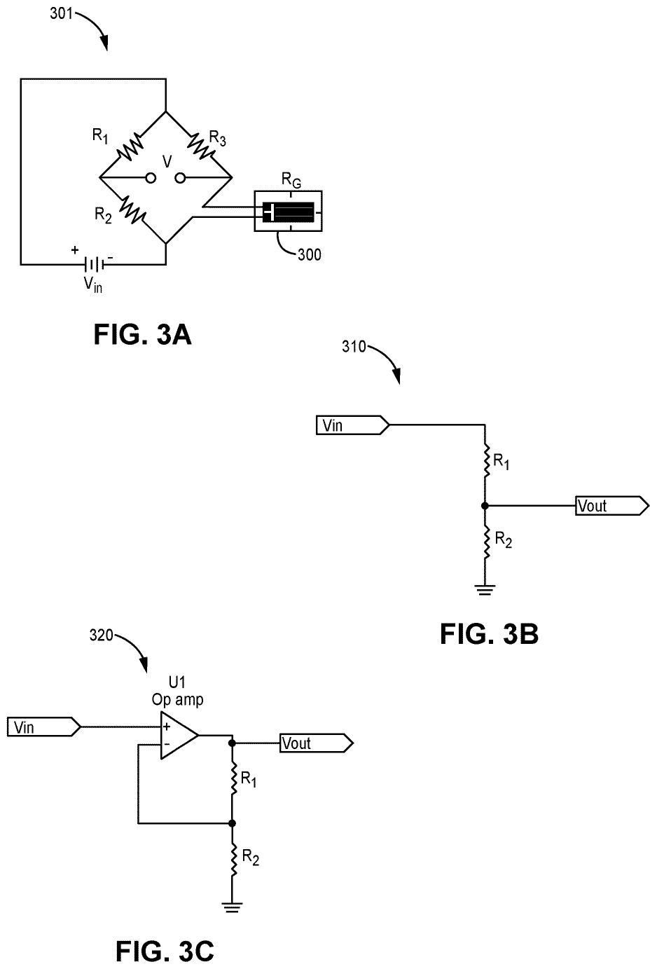

FIG. 3A is a schematic diagram of an exemplary circuit 301 to determine an unknown resistance R_{G} of a hydrogen sensor 300, according to some embodiments. The exemplary circuit 301 is a Wheatstone bridge wherein the unknown resistance R_{G} of the hydrogen sensor 300 is discernable. Resistors R₁, R₂, and R₃ are resistors with known resistance values. Vᵢₙ is the input voltage and V is the output voltage.

FIG. 3B is a schematic diagram of a circuit 310 for converting a resistance change to a voltage (e.g., a voltage divider), according to some embodiments. Vᵢₙ is the input voltage, Vₒᵤₜ is the output voltage, and R₁ and R₂ are resistors. One of the resistors R₁ and R₂ are replaced with an organic hydrogen sensor (e.g., the hydrogen sensor 100). Changes in the organic hydrogen sensor 100 resistance upon exposure to hydrogen would be converted to a change in the output voltage (Vₒᵤₜ).

FIG. 3C is a schematic diagram of a circuit for converting a resistance change to a voltage (e.g., a voltage divider), according to some embodiments. Vᵢₙ is the input voltage, Vₒᵤₜ is the output voltage, Oₚₐₘₚ is an operational voltage amplifier, and R₁ and R₂ are resistors. One of the resistors R₁ and R₂ are replaced with an organic hydrogen sensor (e.g., the hydrogen sensor 100). The hydrogen sensor 100 may produce a small output voltage, for example when connected using the circuits in FIGS. 3A-B, which only results changes of a few millivolts. It may be beneficial to amplify the voltage with the Oₚₐₘₚ such that the voltage is compatible with the operating voltages of the driving circuitry, e.g. analog-to-digital converters. Changes in the organic hydrogen sensor 100 resistance upon exposure to hydrogen would be converted to a change in the output voltage (Vₒᵤₜ).

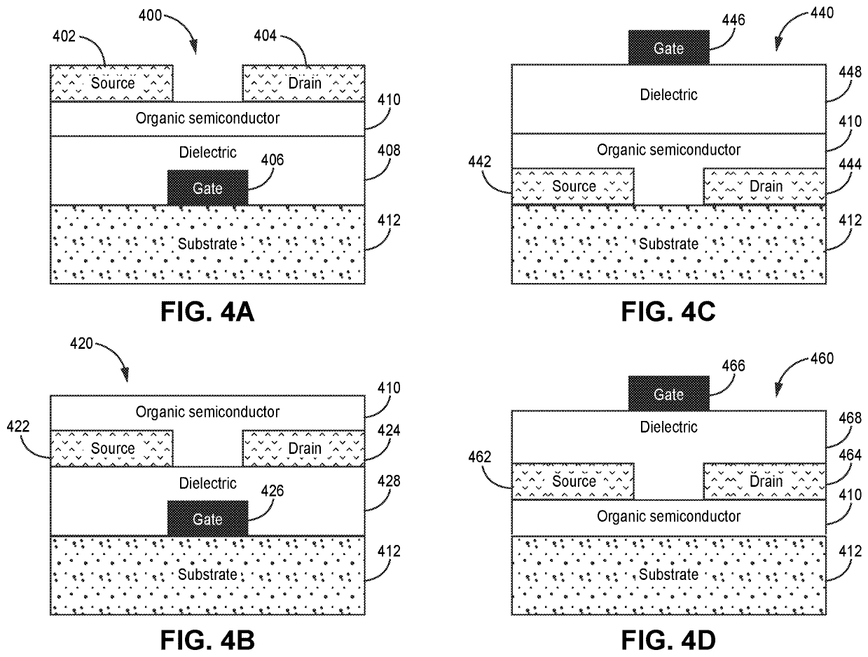

FIGS. 4A-D are diagrammatic views of transistor-type organic hydrogen sensors, according to some embodiments. For instance, FIG. 4A is a field effect transistor 400 having a bottom-gate top-contact (BGTC) configuration. The field effect transistor 400 includes a source 402, a drain 404, a gate 406, a dielectric 408, an organic semiconductor 410, and a substrate 412. The organic semiconductor 410 includes any and/or all features of the organic semiconductor 110 described above in reference to FIG. 1A. The organic semiconductor 410 is a p-type (positive charge transporting) semiconductor. The organic semiconductor 410 is doped upon exposure to oxygen, i.e., the holes of the organic semiconductor 410 receive electrons from oxygen which dopes the organic semiconductor 410. Doping of the organic semiconductor 410 increases conductivity (and decreases resistivity) of the organic semiconductor 410. Exposing the oxygen-doped organic semiconductor 410 to hydrogen de-dopes (deoxidizes) the organic semiconductor 410, thereby reducing the conductivity of the organic semiconductor 410.

The organic semiconductor 410 is located between the gate 406, and the source 402 and the drain 404. Changes to the conductivity (e.g., resistance, capacitance, impedance, etc.) of the organic semiconductor 410 upon exposure to hydrogen changes the input signal to the gate 406, resulting in a change in current (and/or other electrical properties) between the source 402 and the drain 404. In some embodiments, a catalytic material is present within one or more of the source 402, the drain 404, and/or the organic semiconductor 410 (e.g., dispersed within the organic semiconductor 410 and/or deposited atop the organic semiconductor 410).

FIG. 4B is a field effect transistor 420 having a bottom-gate bottom-contact (BGBC) configuration. The field effect transistor 420 includes a source 422, a drain 424, a gate 426, a dielectric 428, the organic semiconductor 410, and the substrate 412. FIG. 4C is a field effect transistor 440 having a top-gate bottom-contact (TGBC) configuration. The field effect transistor 420 includes a source 442, a drain 444, a gate 446, a dielectric 448, the organic semiconductor 410, and the substrate 412. FIG. 4D is a field effect transistor 460 having a top-gate top-contact (TGTC) configuration. The field effect transistor 460 includes a source 462, a drain 464, a gate 466, a dielectric 468, the organic semiconductor 410, and the substrate 412. Other transistor configurations may also be implemented, including for example, field effect transistors with asymmetric contact materials and electrode configuration, bipolar-junction transistors, vertical channel transistors, as well as other variant architectures known to those skilled in the art.

FIGS. 1A-4D illustrate resistive-type and transitive type hydrogen sensors with an organic semiconductor, according to some embodiments. It should be noted that the organic semiconductor can be used to detect the presence of hydrogen and/or hydrogen concentration in capacitive-type and/or optical-type hydrogen sensors. The same principle of operation applies to organic capacitive-type hydrogen sensors and to organic optical-type hydrogen sensors, namely, that the oxygen-doped organic semiconductor is de-doped by hydrogen which thereby changes the conductive properties of the organic semiconductor (decreasing conductivity in the presence of hydrogen). The change of conductivity of the organic semiconductor can be detected via a change in capacitance (e.g., in an organic capacitive-type hydrogen sensor) and/or can be detected via a change in optical properties (e.g., in an optical-type hydrogen sensor). There are other sensor types/methods of measuring a change in conductivity in organic semiconductors, including but not limited to, measuring changes in voltage, current, resistance, capacitance, induction, impedance, optics, acoustics, mechanical properties, electromagnetic signals, etc.

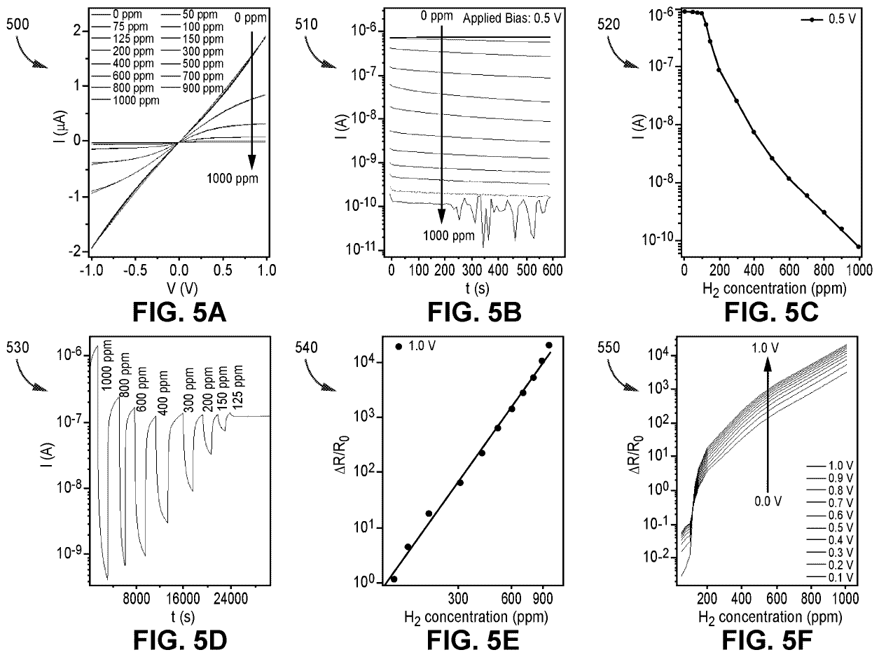

FIGS. 5A-F are results of an exemplary organic hydrogen sensor having interdigitated electrodes (e.g., as shown in FIG. 1A) coated with an organic semiconductor. The organic semiconductor used in these examples includes DPP-DTT polymer (p-type organic semiconductor doapable by oxygen and configured to de-dope in the presence of hydrogen). FIG. 5A is a plot 500 of voltage (V) versus current (μA) as hydrogen concentration (ppm) changes. As the hydrogen concentration increases (0 ppm to 1000 ppm), the current variation is reduced. FIG. 5B is a plot 510 of current variation (A) with time(s) measured at a constant applied voltage of 0.5V. As hydrogen concentration increases (0 ppm to 1000 ppm), the current variation is reduced. FIG. 5C is a plot 520 of current (A) versus hydrogen concentration (H₂ ppm) with a constant applied voltage of 0.5V. As hydrogen concentration increases (0 ppm to 1000 ppm), the current is reduced (from approximately 10⁻⁶ to 10⁻¹⁰ A). FIG. 5D is a plot 530 of real-time(s) current changes (A) at different hydrogen concentrations (ppm). The higher the concentration of hydrogen (e.g., 1000 ppm), the larger the magnitude of current variation. FIG. 5E is a plot 540 of the change in resistance (AR/Ro) of the organic semiconductor versus hydrogen concentration (H₂ ppm). As the hydrogen concentration increases, the change in resistance also increases. It should be noted that the resistance of the organic semiconductor increases with increased H₂ concentration, i.e., the conductivity of the organic semiconductor decreases with increased H₂ concentration. FIG. 5F is a plot 550 of the change in resistance (ΔR/R₀) of the organic semiconductor versus hydrogen concentration (H₂ ppm) at different applied voltages (0.1V to 1.0V).

All of the above plots 500, 510, 520, 530, 540, 550 show that the organic semiconductor (e.g., the organic semiconductor 110 as described in FIG. 1A) exhibits increased resistivity upon exposure to hydrogen. Further, the magnitude of resistance change of the organic semiconductor is a function of the concentration of hydrogen. Similar results have been recorded using C₁₆IDT-BT, poly(3-hexylthiophene-2,5-diyl), and other p-type organic semiconductors doapable by oxygen and configured to de-dope in the presence of hydrogen, as the organic semiconductor material.

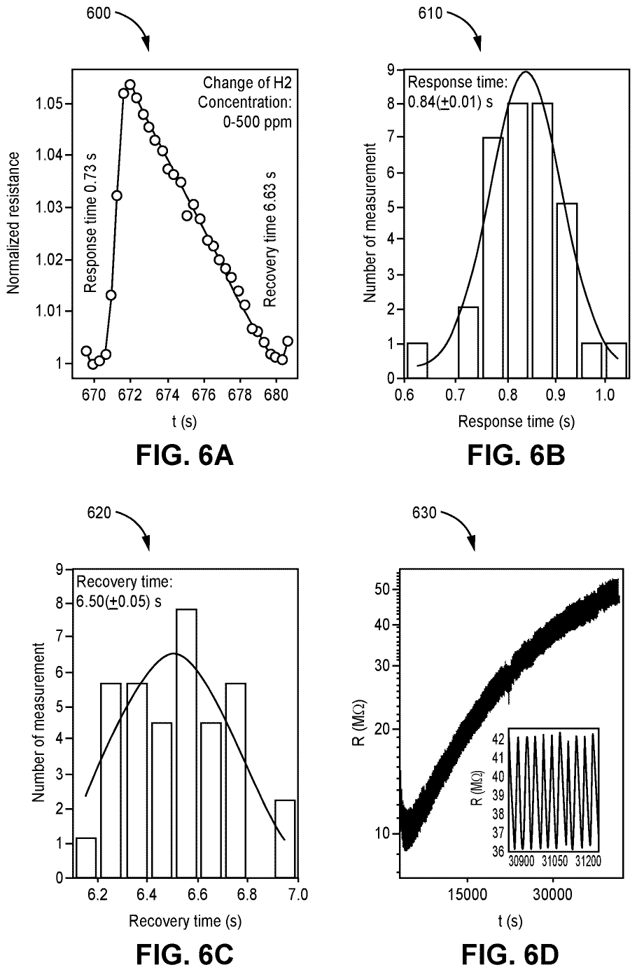

FIGS. 6A-D are results of an exemplary organic hydrogen sensor showing rapid response times and/or recovery times, according to some embodiments. The organic semiconductor used in these examples includes DPP-DTT polymer (p-type organic semiconductor doapable by oxygen and configured to de-dope in the presence of hydrogen). FIG. 6A is a plot 600 of normalized resistance versus time(s) upon exposure of the organic hydrogen sensor to 500 ppm of H₂. The local concentration of hydrogen (at the organic semiconductor) was rapidly changed from 0 ppm to 500 ppm. The normalized resistance of the organic semiconductor peaks after 0.73 seconds of exposure to 500 ppm of H₂. After exposure to the 500 ppm of H₂, the normalized resistance recovers (i.e., resets) to a baseline resistance of 1 after a recover period of 6.63 seconds. FIG. 6B is a bell curve 610 of response times of the hydrogen sensor in response to exposure of 500 ppm of H₂. The response time, or the time for the normalized resistance to peak, is typically less than 1.0 seconds, and in this example, was 0.84 seconds on average. FIG. 6C is a bell curve 620 of the recovery times of the hydrogen sensor in response to exposure of 500 ppm of H₂. The recovery time, or the time for the normalized resistance to reset, is typically less than 7.0 seconds, and in this example, was 6.50 seconds on average. FIG. 6D is a plot 630 of continuous H₂ measurements for 2000 cycles, i.e., the organic hydrogen sensor was exposed to 500 ppm of H₂, the resistance peaked and recovered, and the organic hydrogen sensor was exposed to 500 ppm of H₂, and so on for 2000 cycles.

The organic hydrogen sensor (e.g., the organic hydrogen senor 100, 120, 140, 160, 200, 250, 400, 420, 440, 460 with the organic semiconductor 110, 130, 150, 170, 210, 410) has a rapid response time. As shown in FIGS. 6A-D, the organic hydrogen sensor can detect and quantify the hydrogen concentration in less than 1.0 seconds, and in some cases, between 0.5 seconds and 1.0 seconds. The rapid response time is beneficial, as the organic hydrogen sensor can quickly detect the presence of (potentially hazardous) hydrogen gas, and in some embodiments, initiate an alarm or an emergency system if the hydrogen concentration exceeds a threshold level. As shown in FIGS. 6A-D, the organic hydrogen sensor has a rapid recovery time, which may be beneficial to detect real-time hydrogen concentrations under continuously evolving conditions. The organic hydrogen sensor can undergo thousands of sensing cycles, which is beneficial, as the organic hydrogen sensor can be reliably used over a long period of time.

In some embodiments, the conductivity of the organic hydrogen sensor is partially dependent on humidity of the atmosphere. A humidity sensor can be used in conjunction with, and/or integrated with, the organic hydrogen sensor. In some embodiments, the humidity sensor includes various biomaterials in an active sensing layer to determine humidity levels. For example, interdigitated electrodes (IDEs) are separated from each other and an active sensing layer including a biomaterial is deposited over the interdigitated electrodes. The biomaterial, such as chicken albumen, gelatin, silk fibroin, chitosan, cellulose, keratin or other biomaterials with functional groups such as amine (—NH₂), hydroxyl (—OH), and carboxyl (—COOH), interact with water molecules and trigger the sensing signals.

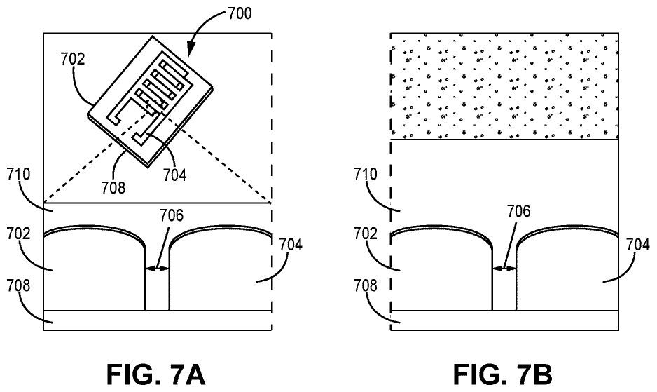

FIG. 7A is a magnified diagrammatic view of a humidity sensor 700, according to some embodiments. The humidity sensor 700 includes a first electrode 702, a second electrode 704, a gap 706 between the first and second electrodes 702, 704, a substrate 708, and a biomaterial layer 710. The biomaterial layer 710 includes one or more of chicken albumen, gelatin, silk fibroin, chitosan, cellulose, keratin or other biomaterials with functional groups such as amine (—NH₂), hydroxyl (—OH), and carboxyl (—COOH), according to some embodiments.

FIG. 7B is a magnified diagrammatic view of a humidity sensor 700 in a humid environment, according to some embodiments. The biomaterial layer 710 swells in response to the humid environment, altering the conductive properties of the biomaterial layer 710 between the first and second electrodes 702, 704.

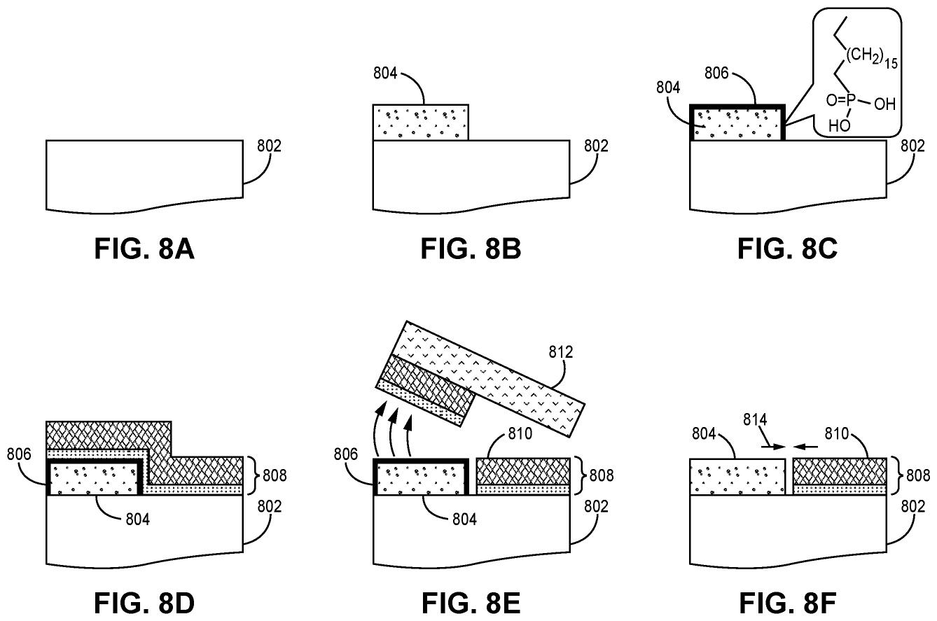

FIG. 8A-F is an exemplary process of creating a nanogap between electrodes, according to some embodiments. The process may be used for manufacturing the interdigitated electrodes of the organic hydrogen sensor and/or manufacturing the interdigitated electrodes of the biomaterial humidity sensor. FIG. 8A is a diagrammatic view of substrate 802. FIG. 8B is a diagrammatic view of the substrate 802 with a first electrode 804 deposited on the substrate 802. The first electrode 804 is formed of a conductive metal and/or a catalytic material. For instance, in one example the first electrode 804 is a thin (~100 nm) metal film (aluminum) deposited and patterned via photolithography. FIG. 8C is a diagrammatic view of the substrate 802 and the first electrode 804 with a self-assembled monolayer (SAM) 806, octadecyl phosphonic acid (ODPA), applied over the first electrode 804. The SAM 806 was chosen to selectively bind to the patterned surfaces of the first electrode 804 selectively but not attach to the remaining substrate 802. FIG. 8D is a diagrammatic view of the substrate 802, the first electrode 804, and the SAM 806 with a second metal film layer 808, 810 deposited over the SAM 806 and the substrate 802. Due to the hydrophobicity of the SAM 806 surface, the subsequently deposited second electrode (including for example, a metal film of ~100 nm gold and ~100 nm titanium) does not adhere to the first electrode 804 but only to the SAM 806 substrate. FIG. 8E is a diagrammatic view of an adhesive 812 partially removing excess material from the second electrode 808. In other words, a mechanical delamination peels off all second electrode areas which overlap with the SAM 806. FIG. 8F is a diagrammatic view of the SAM 806 removed, exposing a nanogap 814. In some embodiments, the SAM 806 is removed via exposing the SAM 806 to an argon plasma. The nanogap 814 reduces the separation distance between electrodes 804, 808, thereby producing a higher electric field between the electrodes 804, 808, resulting in a more sensitive and responsive sensor.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A hydrogen sensor comprising:
a substrate;
a first terminal disposed on the substrate;
a second terminal disposed on the substrate; and
an organic semiconductor electrically coupled to the first terminal, and the second terminal,
wherein conductivity of the organic semiconductor decreases upon exposure to hydrogen.

2. The hydrogen sensor of claim 1, wherein the organic semiconductor is a p-type semiconductor doped upon exposure to oxygen, wherein exposure of the organic semiconductor to hydrogen de-dopes the organic semiconductor of oxygen.

3. The hydrogen sensor of claim 1, wherein an electrical signal is communicated from the first terminal to the second terminal through the organic semiconductor.

4. The hydrogen sensor of claim 1, further comprising:
a catalytic material reactive to hydrogen configured to split hydrogen gas (H₂) into an atomic hydrogen atom (H).

5. The hydrogen sensor of claim 4, wherein one of the first terminal and the second terminal include the catalytic material.

6. The hydrogen sensor of claim 4, wherein the catalytic material is dispersed within the organic semiconductor.

7. The hydrogen sensor of claim 4, wherein the catalytic material is deposited on a surface of the organic semiconductor.

8. The hydrogen sensor of claim 3, wherein one or more characteristics of the electrical signal are measured to determine a change in conductivity of the organic semiconductor.

9. The hydrogen sensor of claim wherein the hydrogen sensor is a resistive-type sensor, wherein the first terminal includes a first electrode and wherein the second terminal includes a second electrode.

10. The hydrogen sensor of claim 1, wherein the hydrogen sensor is a transistor-type sensor, wherein the first terminal includes a source and wherein the second terminal includes a drain.

11. A system for detecting hydrogen, the system including:
an organic hydrogen sensor including:
a first terminal,
a second terminal physically separated from the first terminal by a gap, and
an organic semiconductor electrically coupled to the first terminal and the second terminal;
a processing circuit electrically coupled to the first terminal and the second terminal,
wherein the processing circuit transmits an input signal to one of the first terminal and the second terminal and receives an output signal from one of the first terminal and the second terminal.

12. The system for detecting hydrogen of claim 11, wherein conductivity of the organic semiconductor decreases upon exposure to hydrogen.

13. The system for detecting hydrogen of claim 11, wherein the processing circuit analyzes one or more characteristics of the output signal to determine a change in conductivity of the organic semiconductor, wherein the one or more characteristics of the output signal include a resistance, a voltage, a current, a capacitance, and/or an optical property.

14. The system for detecting hydrogen of claim 13, wherein a decrease in the resistance correlates to an increase in hydrogen gas concentration.

15. The system for detecting hydrogen of claim 11, further comprising:
a catalytic material reactive to hydrogen configured to split hydrogen gas (H₂) into an atomic hydrogen atom (H).

16. The system for detecting hydrogen of claim 15, wherein the catalytic material includes one or more of palladium (Pd), platinum (Pt), gold (Au), and/or nanostructures thereof.

17. The system for detecting hydrogen of claim 11, wherein the organic semiconductor is a p-type semiconductor doped upon exposure to oxygen, wherein exposure of the organic semiconductor to hydrogen de-dopes the organic semiconductor of oxygen.

18. The system for detecting hydrogen of claim 17, wherein the organic semiconductor is formed from conjugated polymers and/or heterocyclic conjugated materials containing thiophene.

19. The system for detecting hydrogen of claim 11, further comprising a humidity sensor the humidity sensor including:
a first electrode,
a second electrode,
a gap separating the first electrode from the second electrode, and
a biomaterial layer filling the gap wherein the biomaterial layer interacts with water molecules, wherein the interaction changes the conductive properties of the biomaterial layer.

20. The system for detecting hydrogen of claim 19, wherein the first electrode and the second electrode are interdigitated electrodes with a nanogap separation therebetween.
